(19) United States
(12) Patent Application Publication
(10) Pub. No.: US 2025/0072896 A1
(43) Pub. Date: Mar. 6, 2025

(54) REPLACEABLE STAPLE CARTRIDGE WITH RETRACTABLE KNIFE

(71) Applicant: Covidien LP, Mansfield, MA (US)

(72) Inventors: Kenneth H. Whitfield, North Haven, CT (US); Roanit A. Fernandes, Hamden, CT (US)

(73) Assignee: Covidien LP, Mansfield, MA (US)

(21) Appl. No.: 18/952,648

(22) Filed: Nov. 19, 2024

Related U.S. Application Data
(63) Continuation of application No. 17/826,271, filed on May 27, 2022, now Pat. No. 12,193,666.

(51) Int. Cl. A61B 17/072

(52) U.S. Cl. CPC A61B 17/07207; A61B 2017/07257; A61B 2017/07278

(57) ABSTRACT

A surgical stapling device includes a tool assembly having a drive assembly, an anvil, and a cartridge assembly. The cartridge assembly includes a replaceable staple cartridge that includes an actuation sled assembly having an actuation sled, a knife bar, and a retractor link. The retractor link is secured to the knife bar such that the actuation sled assembly is movable through a cartridge body of the staple cartridge. The retractor link is movable from a non-deformed condition disengaged from the drive assembly to a deformed condition engaged with the drive assembly to facilitate retraction of the actuation sled assembly after the stapling device is fired.

### CROSS-REFERENCE TO RELATED APPLICATIONS

This application is a continuation of U.S. patent application Ser. No. 17/826,271 filed May 27, 2022, entitled “Replaceable Staple Cartridge with Retractable Knife,” which is incorporated herein by reference in its entirety.

### FIELD

This disclosure is generally related to surgical stapling devices, and more particularly, to surgical stapling devices having replaceable staple cartridges including retractable knives.

### BACKGROUND

Surgical stapling devices configured for endoscopic use are well known and commonly used during surgical procedures to minimize patient trauma and reduce patient recovery times. Typically, endoscopic surgical stapling devices include a tool assembly and a drive assembly that is movable in relation to the tool assembly to actuate the tool assembly. The drive assembly includes a knife bar having a cutting blade for cutting tissue. The tool assembly includes anvil and cartridge assemblies that are coupled to each other by a pivot member and movable in relation to each other between open and clamped positions in response to movement of the drive assembly. The cartridge assembly includes a staple cartridge that has an actuation sled, pushers, and staples. Typically, the drive assembly is movable from a retracted position to an advanced position to advance the actuation sled into contact with the pushers and eject the staples from the staple cartridge.

Some stapling devices include a staple cartridge that can be replaced after each firing of the stapling device to facilitate reuse of the stapling device. In such devices, a knife is typically included on the drive assembly and is reused. Other stapling devices include a reload assembly that includes a staple cartridge and a drive assembly that can be replaced after each firing of the stapling device to facilitate reuse of the stapling device. The use of a reload assembly provides a new cutting blade for each firing of the stapling device to maintain the quality of cutting. However, replacement of the drive assembly after each firing of the stapling device is costly.

A continuing need exists for a surgical stapling device that can in a cost-effective manner provide a sharpened cutting blade through multiple firings of the stapling device.

### SUMMARY

This disclosure is directed to a surgical stapling device that includes a cartridge assembly having a staple cartridge that is replaceable to facilitate reuse of the stapling device. The staple cartridge includes an actuation sled assembly having an actuation sled, a knife bar, and a retractor link. The retractor link is secured to the knife bar such that the actuation sled assembly is movable through a cartridge body of the staple cartridge. The retractor link is movable from a non-deformed condition disengaged from the drive assembly to a deformed condition engaged with the drive assembly to facilitate retraction of the actuation sled assembly or the knife bar after the stapling device is fired.

Aspects of this disclosure are directed to an actuation sled assembly that includes an actuation sled, a knife bar, and a retractor link. The actuation sled includes a central portion and spaced wedge members positioned on opposite sides of the central portion. The knife bar engaged with or fixedly secured to the central portion of the actuation sled and includes a cutting edge positioned above the actuation sled. The retractor link is formed of a resilient material and has a proximal portion and a distal portion. The distal portion defines a longitudinal axis. The retractor link has a non-deformed condition and a deformed condition. In the non-deformed condition, the proximal portion of the retractor link bends outwardly from the longitudinal axis defined by the distal portion of the retractor link, and in the deformed condition, the proximal portion of the retractor link is substantially aligned with the longitudinal axis of the distal portion of the retractor link.

In aspects of the disclosure, the proximal portion of the retractor link includes an engaging member.

In some aspects of the disclosure, the engaging member includes a bend formed on the proximal portion of the retractor link.

In certain aspects of the disclosure, the engaging member includes a protrusion formed on the proximal portion of the retractor link.

In aspects of the disclosure, the recess is formed in the working member of the drive assembly.

In some aspects of the disclosure, the recess is formed in the flexible drive beam of the drive assembly.

In certain aspects of the disclosure, the retractor link is formed from spring steel.

In some aspects of the disclosure, the knife bar includes a base portion, and the central portion of the actuation sled is molded about the base portion of the knife bar to secure the knife bar to the actuation sled.

In certain aspects of the disclosure, the knife bar includes a body portion that defines a longitudinally extending recess and the distal portion of the retractor link is secured within the longitudinally extending recess.

Other aspects of the disclosure are directed to a surgical stapling device including a tool assembly and a drive assembly. The tool assembly includes an anvil and a cartridge assembly. The cartridge assembly includes a staple cartridge having a cartridge body, pushers, staples, and an actuation sled assembly. The cartridge body defines a knife slot and staple receiving pockets positioned on each side of the knife slot. Each of the staple receiving pockets receives one of the staples and one of the pushers. The actuation sled assembly includes an actuation sled, a knife bar, and a retraction link. The actuation sled includes a central portion and spaced wedge members positioned on opposite sides of the central portion. The central portion is received and movable within the knife slot. The knife bar includes a cutting edge that is positioned above the actuation sled and extends from the knife slot. The retractor link is formed of a resilient material and has a proximal portion and a distal portion. The distal portion of the retractor link defines a longitudinal axis. The retractor link has a non-deformed condition and a deformed condition. In the non-deformed condition, the proximal portion of the retractor link bends outwardly from the longitudinal axis of the distal portion of the retractor link and outwardly of the knife slot, and in the deformed condition, the proximal portion of the retractor link is substantially aligned with the longitudinal axis of the distal portion of the retractor link. The proximal portion of the retractor link includes an engaging member. The drive assembly includes a flexible drive beam and a working end that is secured to the flexible drive beam. The drive assembly is movable between retracted and advanced positions in relation to the tool assembly to eject the staples from the cartridge body. The drive assembly defines a recess that receives the engaging member of the retractor link when the retractor link is in the deformed condition to couple the knife bar of the actuation sled assembly to the drive assembly.

In aspects of the disclosure, the stapling device includes an adapter assembly having a proximal portion and a distal portion that supports the tool assembly.

In some aspects of the disclosure, the stapling device includes a handle assembly that is coupled to the proximal portion of the adapter assembly.

In certain aspects of the disclosure, the cartridge assembly is coupled to the anvil to facilitate movement of the tool assembly between unclamped and clamped positions.

In aspects of the disclosure, the drive assembly is movable from the retracted position to a drive assembly clamped position to move the tool assembly from the unclamped position to the clamped position.

In some aspects of the disclosure, the drive assembly is movable independently of the actuation sled assembly between the retracted position and the drive assembly clamped position.

Other features of the disclosure will be appreciated from the following description.

### BRIEF DESCRIPTION OF THE DRAWINGS

Various aspects of the disclosed surgical stapling device are described herein below with reference to the drawings, wherein:

FIG. 1 is a side perspective view of a surgical stapling device according to aspects of the disclosure with a tool assembly of the stapling device in an open position;

FIG. 2 is an enlarged view of the indicated area of detail of FIG. 1;

FIG. 3 is a side perspective view of the tool assembly of the stapling device shown in FIG. 1 with a staple cartridge of a cartridge assembly of the tool assembly separated from a channel of the cartridge assembly;

FIG. 4 is a side perspective exploded view of the staple cartridge of the cartridge assembly of the tool assembly shown in FIG. 3;

FIG. 5 is an enlarged view of the indicated area of detail shown in FIG. 4 illustrating an actuation sled assembly of the staple cartridge shown in FIG. 4;

FIG. 6A is a side perspective, exploded view of the actuation sled assembly shown in FIG. 5;

FIG. 6B is a perspective view from a proximal end of the actuation sled assembly shown in FIG. 5;

FIG. 7 is a cross-sectional view taken along section line 7-7 of FIG. 6B;

FIG. 8 is a bottom, perspective view of the staple cartridge of the cartridge assembly shown in FIG. 4;

FIG. 9 is a cross-sectional view taken along section line 9-9 of FIG. 8;

FIG. 10 is a perspective view of a proximal portion of the staple cartridge shown in FIG. 8 with the actuation sled assembly shown in FIG. 8;

FIG. 11 is a side view of the tool assembly shown in FIG. 2 with the tool assembly moved to the clamped position and the channel member removed;

FIG. 12 is an enlarged view of the indicated area of detail shown in FIG. 11;

FIG. 13 is a side perspective view of a drive assembly and the actuation sled assembly of the surgical stapling device shown in FIG. 1 in retracted positions;

FIG. 14 is a cross-sectional view taken along section line 14-14 of FIG. 12;

FIG. 15 is a side cutaway view of a proximal portion of the tool assembly with the channel of the cartridge assembly removed as the tool assembly is fired;

FIG. 16 is a cross-sectional view taken along section line 16-16 of FIG. 15;

FIG. 17 is a side cutaway view of the proximal portion of the tool assembly with the channel of the cartridge assembly removed as the drive assembly is returned to its retracted position after the stapling device has been fired;

FIG. 18 is a cross-sectional view taken along section line 18-18 of FIG. 17;

FIG. 19 is a side perspective view from above of the proximal portion of the staple cartridge after the staple cartridge has been fired and removed from the channel of the cartridge assembly;

FIG. 20 is a side perspective view of an alternate version of the actuation sled assembly of the surgical stapling device shown in FIG. 1;

FIG. 21 is a side perspective, exploded view of a knife assembly of the actuation sled assembly shown in FIG. 20;

FIG. 22 is a side perspective view of an alternate version of the drive assembly of the surgical stapling device shown in FIG. 1 in their retracted positions;

FIG. 23 is a side perspective view of an alternate version of the actuation sled assembly of the surgical stapling device shown in FIG. 1;

FIG. 24 is an exploded view of the actuation sled assembly of FIG. 23;

FIG. 25 is a cross-sectional view taken along section line 25-25 of FIG. 23;

FIG. 26 is a cross-sectional view taken through the distal portion of the tool assembly with the stapling device in the clamped and fired position and the drive assembly in the advanced position;

FIG. 27 is a cross-sectional view taken through the distal portion of the tool assembly with the stapling device in the clamped and fired position as the drive assembly moves from the advanced position back towards the retracted position with the knife bar coupled to the drive assembly; and

FIG. 28 is a cross-sectional view taken through the proximal portion of the tool assembly with the stapling device in the clamped and fired position and the drive assembly in the retracted position with the knife bar uncoupled from the drive assembly.

### DETAILED DESCRIPTION

The disclosed surgical stapling device will now be described in detail with reference to the drawings in which like reference numerals designate identical or corresponding elements in each of the several views. However, it is to be understood that disclosed aspects of the surgical stapling device are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure.

In this description, the term “proximal” is used generally to refer to that portion of the device that is closer to a clinician during use of the device in its customary fashion, while the term “distal” is used generally to refer to that portion of the device that is farther from the clinician during use of the device in its customary fashion. In addition, directional terms such as front, rear, upper, lower, top, bottom, and similar terms are used to assist in understanding the description and are not intended to limit the disclosure. Further, the term “clinician” is used generally to refer to medical personnel including doctors, nurses, surgeons, and support personnel.

The disclosure is directed to a surgical stapling device that includes a tool assembly having a drive assembly, an anvil, and a cartridge assembly. The cartridge assembly includes a replaceable staple cartridge that includes an actuation sled assembly having an actuation sled, a knife bar, and a retractor link. The retractor link is secured to the knife bar such that the actuation sled assembly is movable through a cartridge body of the staple cartridge. The retractor link is movable from a non-deformed condition disengaged from the drive assembly to a deformed condition engaged with the drive assembly to facilitate retraction of the actuation sled assembly after the stapling device is fired.

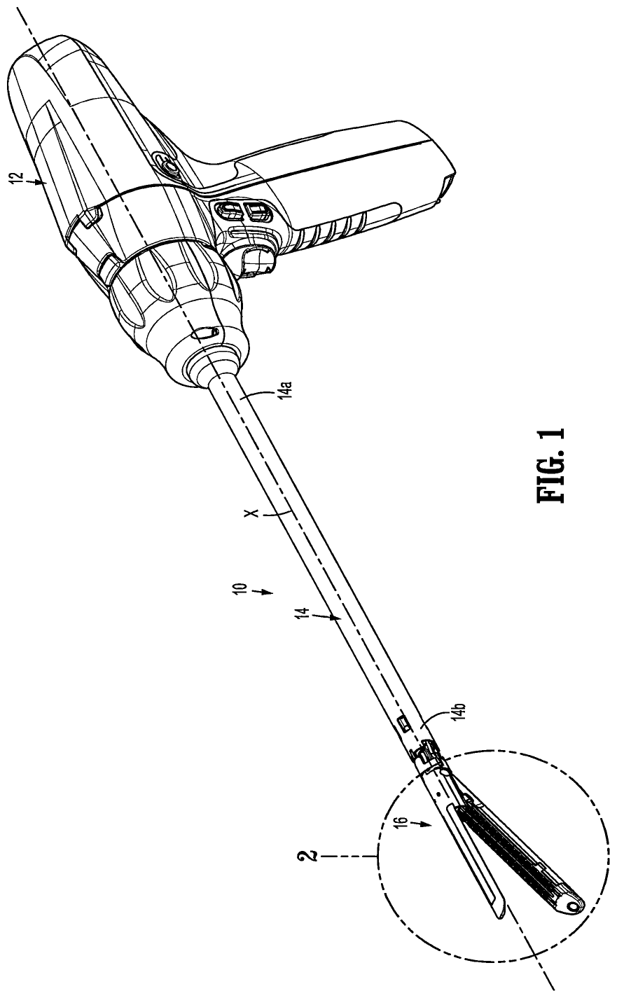

FIG. 1 illustrates a surgical stapling device shown generally as stapling device 10 that includes a handle assembly 12, an adapter assembly 14, and a tool assembly 16. The handle assembly 12 is powered and includes a stationary handgrip 18 and actuation buttons 20. The actuation buttons 20 are operable to actuate various functions of the tool assembly 16 via the adapter assembly 14, i.e., approximation of the tool assembly 16, and firing of the tool assembly 16. In certain aspects of the disclosure, the handle assembly 12 includes a motor (not shown) batteries (not shown), and circuitry that couples the batteries to the motor to operate the stapling device 10. Although the stapling device 10 is illustrated as a powered stapling device, it is envisioned that the advantages of this disclosure are suitable for use with manually powered surgical stapling devices as well as robotically controlled stapling devices. U.S. Pat. No. 5,865,361 describes a stapling device that includes exemplary aspects of a manually powered stapling device.

The adapter assembly 14 defines a longitudinal axis “X” and includes a proximal portion 14a and a distal portion 14b. The proximal portion 14a of the adapter assembly 14 is coupled to the handle assembly 12, and the distal portion 14b of the adapter assembly 14 is coupled to the tool assembly 16.

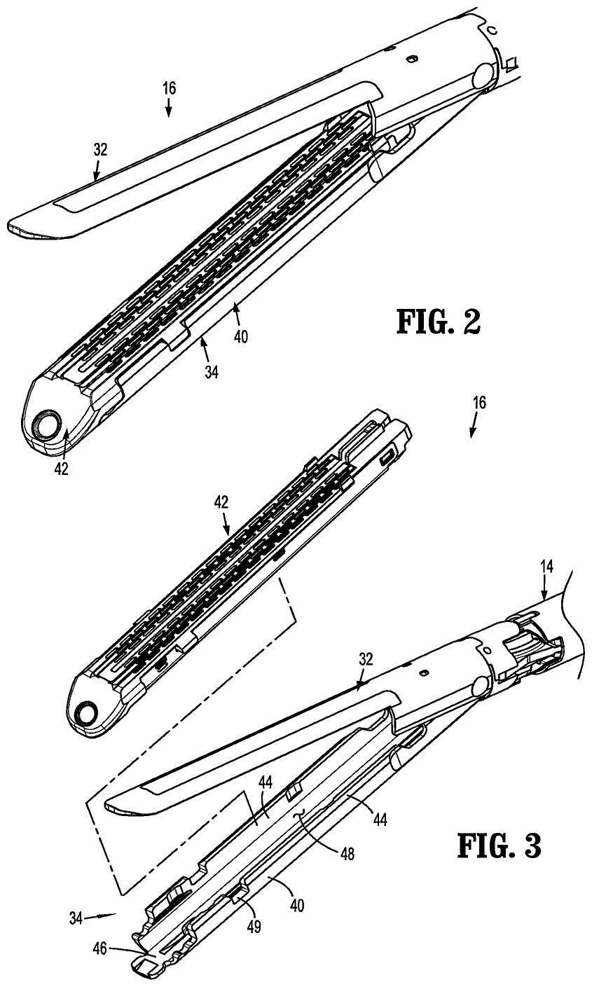

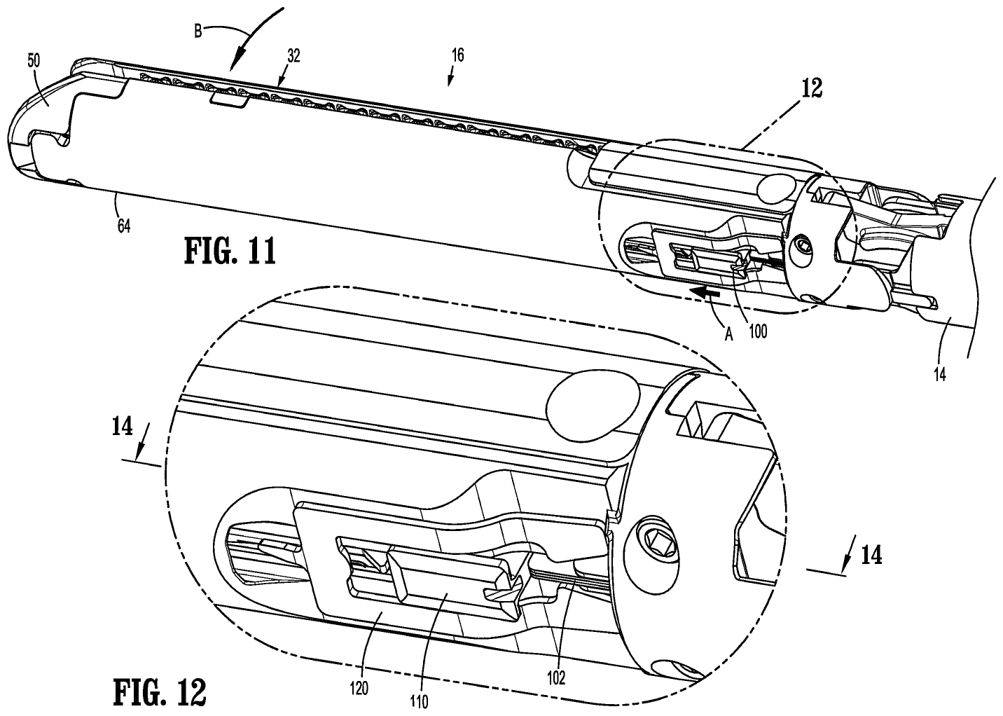

FIGS. 2 and 3 illustrate the tool assembly 16 which includes an anvil 32 and a cartridge assembly 34. In aspects of the disclosure, the cartridge assembly 34 is pivotably supported in relation to the anvil 32 and the adapter assembly 14 such that the tool assembly 16 is movable between an open position (FIG. 2) and a clamped position (FIG. 11). Alternately, it is envisioned that the anvil 32 may be pivotably supported in relation to the cartridge assembly 34 and the adapter assembly 14.

FIGS. 2-5 illustrate the cartridge assembly 34 which includes a channel member 40 and a staple cartridge 42. The channel member 40 includes side walls 44 and a bottom wall 46 that define a cavity 48. The staple cartridge 42 is removably received within the cavity 48 of the channel member 38 and is replaceable to facilitate reuse of the stapling device 10. Each of the side walls 44 defines a recess 49 (FIG. 3) on an upper edge of the side wall 44.

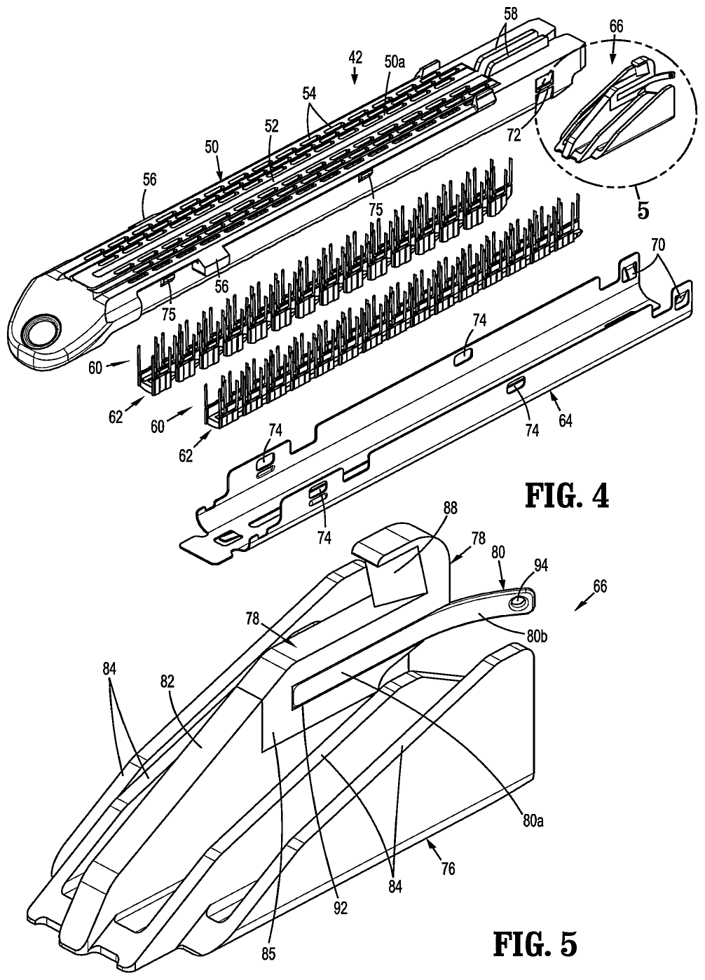

FIG. 4 illustrates the staple cartridge 42 which includes a cartridge body 50 that defines a knife slot 52 and staple receiving pockets 54 positioned on each side of the knife slot 52. In aspects of the disclosure, the knife slot is centrally located along a longitudinal axis of the cartridge body 50. In aspects of the disclosure, the staple receiving pockets 54 are aligned in two or more rows on opposite sides of the knife slot 52. The cartridge body 50 includes laterally extending protrusions 56 that are received within the recesses 49 (FIG. 3) of the channel member 40 (FIG. 3) to properly position the staple cartridge 40 within the cavity 46 of the channel member 38. The cartridge body 50 also includes knife guards 58 that are positioned on opposite sides of the knife slot 52 at a proximal portion of the cartridge body 50.

The staple cartridge 42 includes staples 60, pushers 62, a staple guard 64, and an actuation sled assembly 66. The staples 60 are supported on the pushers 62 and received within the staple receiving pockets 54 of the cartridge body 50. The staple guard 64 is U-shaped and is secured to the bottom of the cartridge body 50 to retain the staples 60 and pushers 62 within the cartridge body 50. In aspects of the disclosure, the staple guard 64 includes resilient fingers 70 that are received in snap-fit fashion in recesses 72 in the cartridge body 50 to secure a proximal portion of the staple guard 64 to the cartridge body 50. The staple guard 64 may also include openings 74 that receive protrusions 75 on the cartridge body 50 to retain the staple guard 64 on the cartridge body 50.

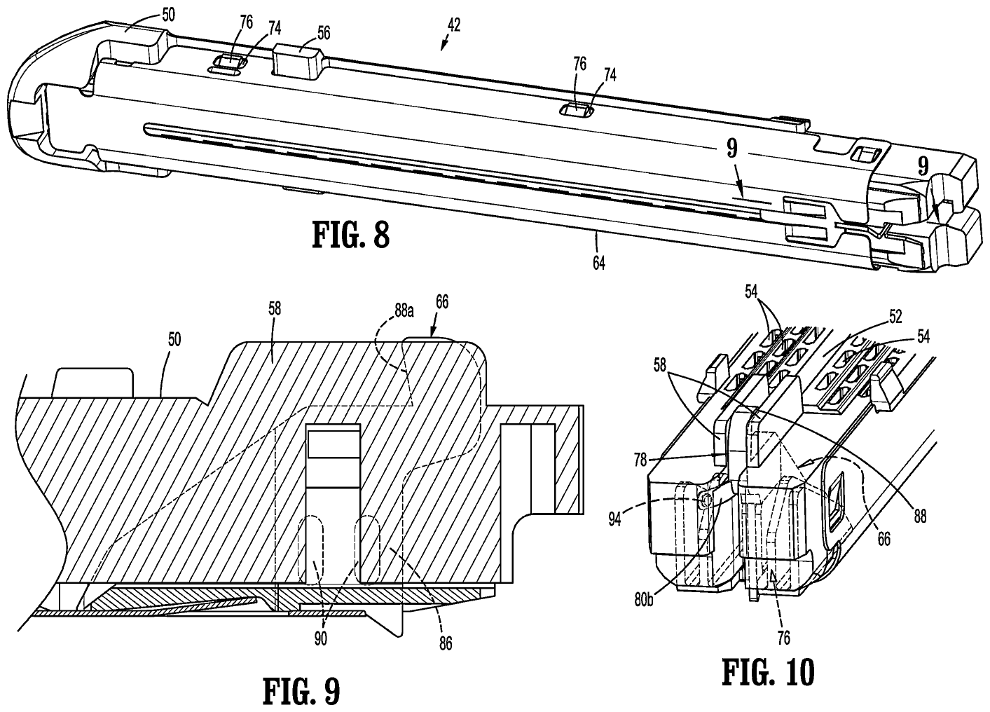

FIGS. 5-12 illustrate the actuation sled assembly 66 which includes an actuation sled 76, a knife bar 78, and a retractor link 80. The actuation sled assembly 66 is received in the cartridge body 50 of the staple cartridge 42 and is movable within the cartridge body 50 between sled retracted and sled advanced positions. The actuation sled 76 includes a central portion 82 and spaced wedge members 84 that are positioned on opposite sides of the central portion 82. The wedge members 84 are configured to engage and lift the pushers 62 as the actuation sled assembly 66 is moved from the sled retracted position to the sled advanced position to eject the staples 60 from staple receiving pockets 54 of the cartridge body 50. The central portion 82 of the actuation sled 76 is received in the knife slot 52 of the cartridge body 50 and is movable through the knife slot 52 to limit the actuation sled assembly 66 to linear movement.

The knife bar 78 is fixedly secured to the central portion 82 of the actuation sled 76 and includes a body portion 85, a base portion 86, and knife 88. The body portion 85 of the knife bar 78 is received in and movable through the knife slot 52. The knife 88 is supported on an upper surface of the body portion 85 and includes a distally facing cutting edge 88a. The cutting edge 88a extends upwardly from the body portion 85 to a position above a tissue engaging surface 50a of the cartridge body 50 and is shielded by the knife guards 58 (FIG. 4). The knife guards 58 may be integrally formed with the cartridge body 50 or secured thereto. The base portion 86 extends downwardly from the body portion 85 and is secured to the central portion 82 of the actuation sled 76. In aspects of the disclosure, the base portion 86 defines two openings 90 that receive portions of the central portion 82 of the actuation sled 76. In certain aspects of the disclosure, the actuation sled 76 is formed of plastic and the knife bar 78 is formed from metal, and the actuation sled 76 is molded about the base portion 86 of the knife bar 78.

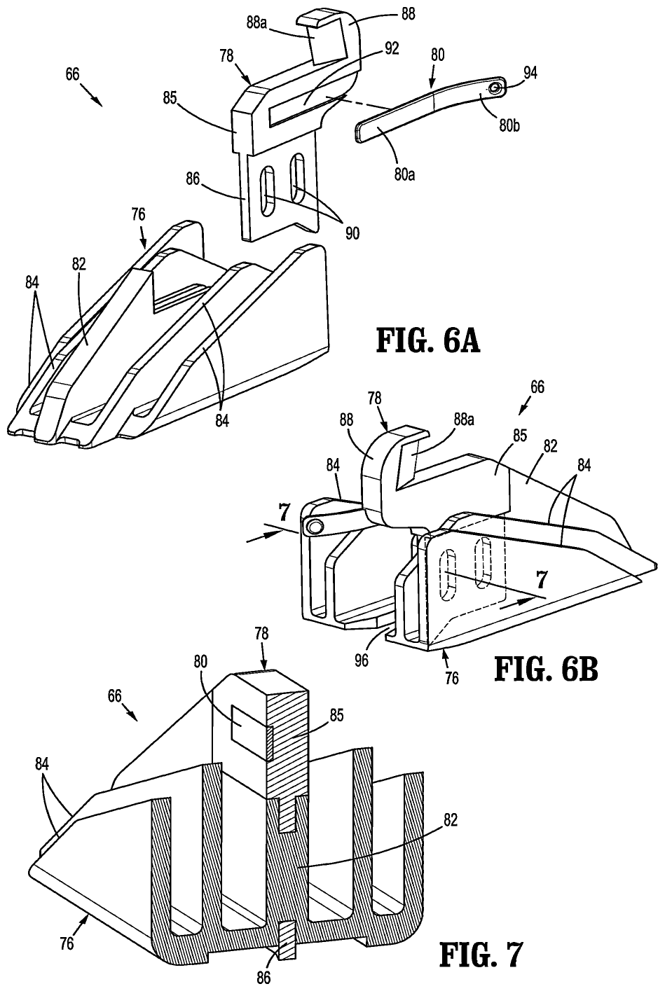

The retractor link 80 is secured to the body portion 85 of the knife bar 78 in cantilevered fashion. In aspects of the disclosure, the retractor link 80 is formed of a resilient material, e.g., spring steel, and includes a distal portion 80a and a proximal portion 80b. The proximal portion 80b of the retractor link 80 includes an engaging member which may be in the form of an inwardly extending protrusion 94. The distal portion 80a defines a longitudinal axis and is secured to the central portion 82 of the knife bar 78, and the proximal portion 80b extends proximally from the actuation sled 76. In aspects of the disclosure, the body portion 85 of the knife bar 78 defines a longitudinally extending recess 92 (FIG. 6A), and the distal portion 80a of the retractor link 80 is secured within the recess 92. The retractor link 80 is movable between a non-deformed condition and a deformed condition. In the non-deformed condition, the proximal portion of the retractor link 80 bends outwardly from the longitudinal axis defined by the distal portion 80 of the retractor link 80 and outwardly of the knife slot 52 of the cartridge body 50. When the actuation sled assembly 66 is advanced through the knife slot 52, the proximal portion 80b of the retractor link 80 engages an inner wall of the cartridge body 50 defining the knife slot 52 and is moved to the deformed condition in which the distal and proximal portions 80a, 80b of the retractor link 80 are longitudinally aligned. The actuation sled 76 of the actuation sled assembly 66 includes a proximal portion that defines a centrally located cutout 96 (FIG. 6B).

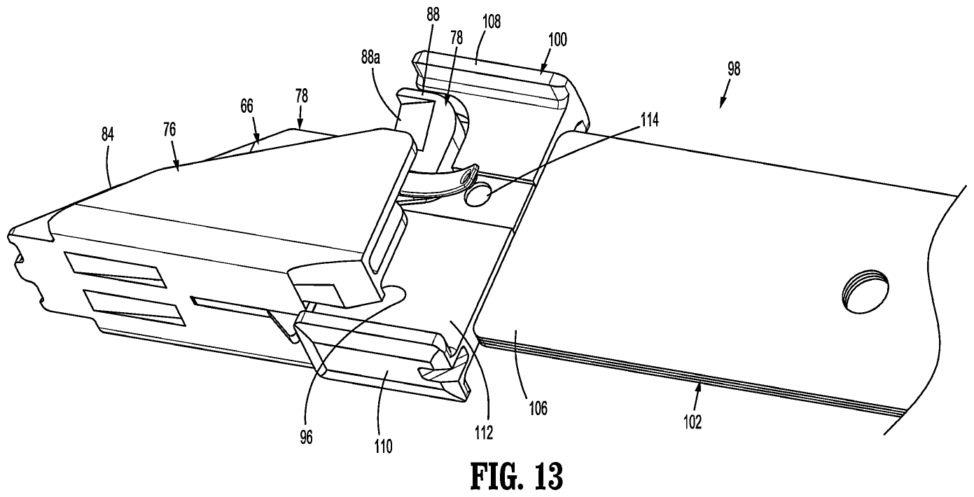

The stapling device 10 includes a drive assembly 98 illustrated in FIG. 13 which includes a working member 100 and a flexible drive beam 102. The working member 100 of the drive assembly 98 is partly received in the cutout 96 of the actuation sled 76 when the drive assembly 98 is in a clamped position and the actuation sled assembly 66 is in a retracted position. The drive assembly 98 is movable from a retracted position, through a drive assembly clamped position, to advanced position and includes a flexible drive beam 102 that has a proximal portion (not shown) and a distal portion 106 that is secured to the working member 100. In aspects of the disclosure, the flexible drive beam 102 is formed from stacked laminates. Alternately, other configurations of the flexible drive beam 102 are envisioned. When the drive assembly 98 is moved from the retracted position to the drive assembly clamped position, the working member 100 moves independently of the actuation sled assembly 66 to move the tool assembly 16 (FIG. 1) from the unclamped position to the clamped position.

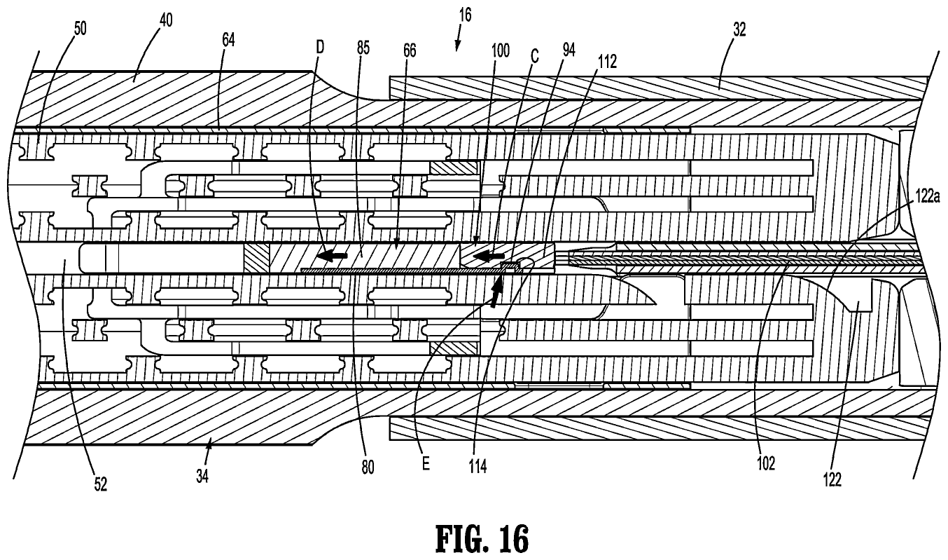

The working member 100 of the drive assembly 98 has an I-shaped configuration and includes a first beam 108, a second beam 110, and a vertical strut 112 that connects the first beam 108 to the second beam 110. The vertical strut 112 is aligned with the knife slot 52 (FIG. 16) in the cartridge body 50 and is positioned to engage the actuation sled assembly 66. When the drive assembly 98 moves from the retracted position to the clamped position in the direction of arrow “A” in FIG. 11, the first and second beams 108, 110 engage the anvil 32 and the cartridge assembly 34 to pivot the cartridge assembly 34 in the direction of arrow “B” in FIG. 11. In the clamped position of the drive assembly 98, the working member 100 of the drive assembly 98 is received in the cutout 96 of the actuation sled 76 of the actuation sled assembly 66 and is in close approximation or abutting relation to the actuation sled assembly 66. When the drive assembly 98 is moved from the clamped position towards the advanced position, the vertical strut 112 of the working member 100 of the drive assembly 98 engages and advances the actuation sled assembly 66 through the knife slot 52 to move the actuation sled assembly 66 between the sled retracted and advanced positions.

The vertical strut 112 of the working member 100 of the drive assembly 98 defines a recess or opening 114. When the actuation sled assembly 66 is moved by the drive assembly 98 from the sled retracted position towards the sled advanced position, the retractor link 80 of the actuation sled assembly 66 moves with the actuation sled assembly 66 into the knife slot 52 of the cartridge body 50. As the proximal portion 80b of the retractor link 80 moves into the knife slot 52 of the cartridge body 50, engagement of the proximal portion 80b of the retractor link 80 with the cartridge body 50 moves the retractor link 80 from the undeformed condition to the deformed condition. When this occurs, the protrusion 94 of the retractor link 80 is received in the recess or opening 114 formed in the vertical strut 112 to secure the actuation sled assembly 66 to the working member 100 of the drive assembly 98.

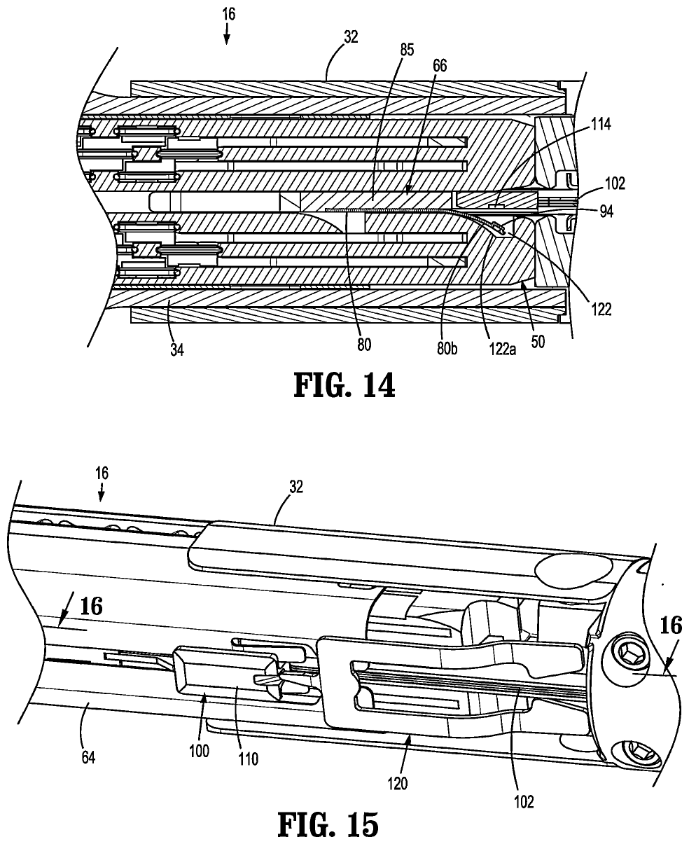

FIGS. 13-15 illustrate the tool assembly 16 with the drive assembly 98 in the drive assembly clamped position and the actuation sled assembly 66 in the sled retracted position. Although not described in detail herein, the cartridge assembly 34 includes a lockout member 120 that prevents refiring of the tool assembly 16 with a spent staple cartridge. The lockout member 120 does not form part of this disclosure and will not be described in further detail herein. When the drive assembly 98 is in the clamped position, and the actuation sled assembly 66 is in the sled retracted position, the retractor link 80 is in its non-deformed condition in which the proximal portion 80b of the retractor link 80 is bent outwardly of the knife slot 52 of the cartridge body 50. To accommodate the proximal portion 80b of the retractor link 80 when the retractor link 80 is in the non-deformed condition, the cartridge body 50 defines a pocket 122 (FIG. 14) that includes a curved wall 122a that has a curvature that corresponds to the curvature of the proximal portion 80b of the retractor link 80 in the non-deformed condition.

FIG. 16 illustrates the tool assembly 16 as the drive assembly 98 is moved from the clamped position towards the advanced position to move the actuation sled assembly 66 from the sled retracted position (FIG. 14) towards the sled advanced position. When the drive assembly moves in the direction of arrow “C” from the drive assembly clamped position towards the advanced position, the working member 100 of the drive assembly 98 moves the actuation sled assembly 66 in the direction of arrow “D”. As the proximal portion 80b of the retractor link 80 moves into the knife slot 52 of the cartridge body 50, the proximal portion 80b of the retractor link 80 engages the curved wall 122a of the cartridge body 50 and is moved from the undeformed condition to the deformed condition in the direction of arrow “E”. As the retractor link 80 moves to the deformed condition, the protrusion 94 of the retractor link 80 is received within the recess 114 formed in the vertical strut 112 of the working member 100 to couple the actuation sled assembly 66 to the working member 100 of the drive assembly 98. As the drive member 98 moves towards the advanced position to move the actuation sled assembly 66 towards the sled advanced position, the spaced wedge members 84 (FIG. 13) of the actuation sled 76 engage the pushers 62 (FIG. 4) of the staple cartridge 42 to eject the staples 60 from the staple receiving slots 54 of the cartridge body 50 into the anvil 32.

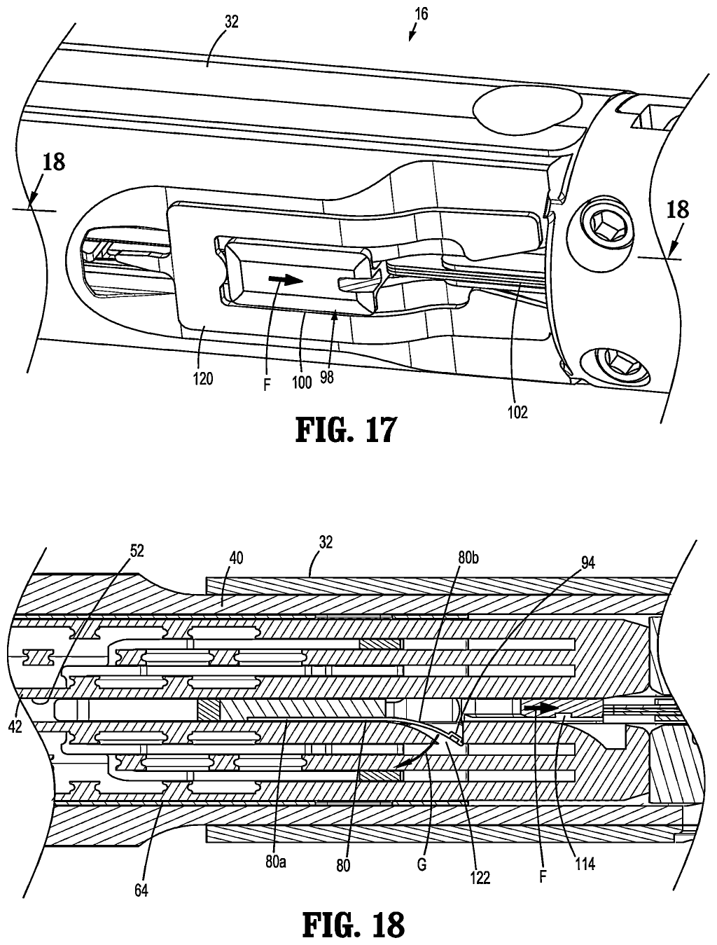

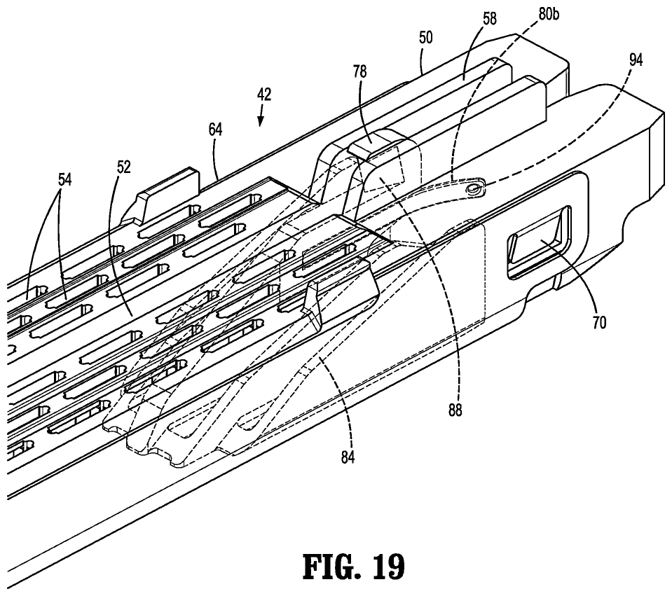

FIGS. 17 and 18 illustrate the tool assembly 16 as the drive assembly 98 is returned from the advanced position to the retracted position in the direction of arrow “F”. As described above, the protrusion 94 of the retractor link 80 is received in the recess or opening 114 in the vertical strut 112 of the working member 100 of the drive assembly 98 to couple the actuation sled assembly 66 to the drive assembly 98. Thus, as the drive assembly 98 returns from the advanced position to the retracted position, the actuation sled assembly 66 also moves from the sled advanced position to the sled retracted position. When the actuation sled assembly 66 returns to the sled retracted position and the drive assembly 98 is returned to the drive assembly clamped position, the proximal portion 80b of the retractor link 80 becomes aligned with the pocket 122 in the cartridge body 50. When this occurs, the proximal portion 80b of the retractor link 80 returns to the non-deformed position in the direction of arrow “G” in FIG. 18 to uncouple the actuation sled assembly 66 from the drive assembly 98. After the actuation sled assembly 66 is uncoupled from the drive assembly 98, the drive assembly 98 continues to move towards the retracted position independently of the actuation sled assembly 66. In this position, the spent staple cartridge 42 (FIG. 19) can be removed from channel member 34 (FIG. 3) and replaced with a fresh staple cartridge to facilitate reuse of the stapling device 10 (FIG. 1).

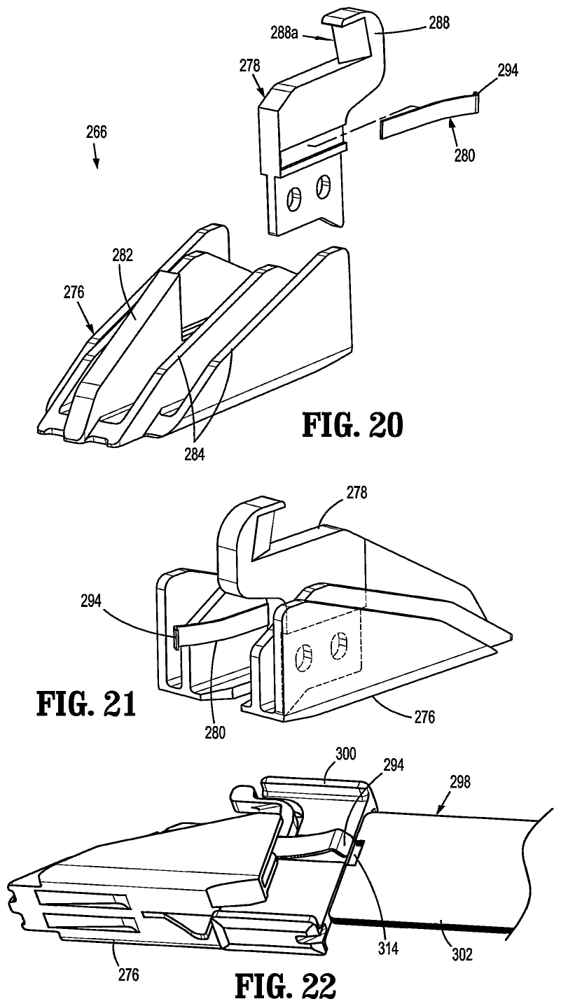

FIGS. 20-22 illustrate an alternate version of the actuation sled assembly shown generally as actuation sled assembly 266 and the drive assembly shown generally as drive assembly 298 (FIG. 22). The actuation sled assembly 266 is like actuation sled assembly 66 (FIG. 6A) and includes an actuation sled 276, a knife bar 278, and a retractor link 280. The knife bar 278 includes a knife 288 having a distally facing cutting edge 288a. The actuation sled assembly 266 is received in the cartridge body 50 (FIG. 4) of the staple cartridge 42 and is movable within the cartridge body 50 between sled retracted and sled advanced positions. The actuation sled 276 includes a central portion 282, and spaced wedge members 284 that are positioned on opposite sides of the central portion 282. The wedge members 284 are configured to engage and lift the pushers 62 (FIG. 4) as the actuation sled assembly 266 is moved from the sled retracted position to the sled advanced position to eject the staples 60 (FIG. 4) from staple receiving pockets 54 of the cartridge body 50. The central portion 282 of the actuation sled 276 is received in the knife slot 52 (FIG. 4) of the cartridge body 50 and is movable through the knife slot 52 to limit the actuation sled assembly 266 to linear movement. The actuation sled assembly 266 differs from the actuation sled assembly 66 (FIG. 6A) in that the retractor link 280 includes a proximal portion that includes an engaging member in the form of a bend 294.

The drive assembly 298 is like the drive assembly 98 and includes a working member 300 and a flexible drive beam 302. The working member 300 and the flexible drive beam 302 are as described above regarding working member 100 and flexible drive beam 102 except that a distal portion of the flexible drive beam 302 defines a recess or opening 314. In some aspects of the disclosure, the recess or opening 314 may also be formed in the vertical strut 312 of the working member 300.

The actuation sled assembly 266 and the drive assembly 298 function in the same manner as the actuation sled assembly 66 and the drive assembly 98. More specifically, when the drive assembly 298 moves from the drive assembly clamped position towards the advanced position to move the actuation sled assembly 266 from the sled retracted position towards the sled advanced position, the retractor link 280 moves into the knife slot 52 (FIG. 4) and is deformed from the undeformed condition to the deformed condition. When this occurs, the bend 294 is received in the opening 314 defined in the drive assembly 298 to couple the actuation sled assembly 266 to the drive assembly 298.

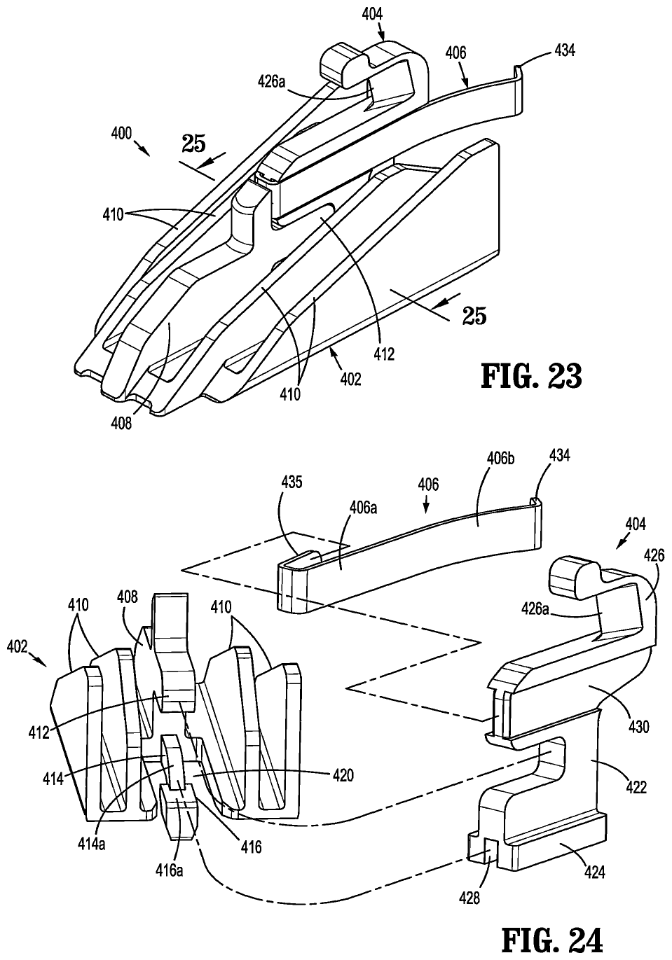

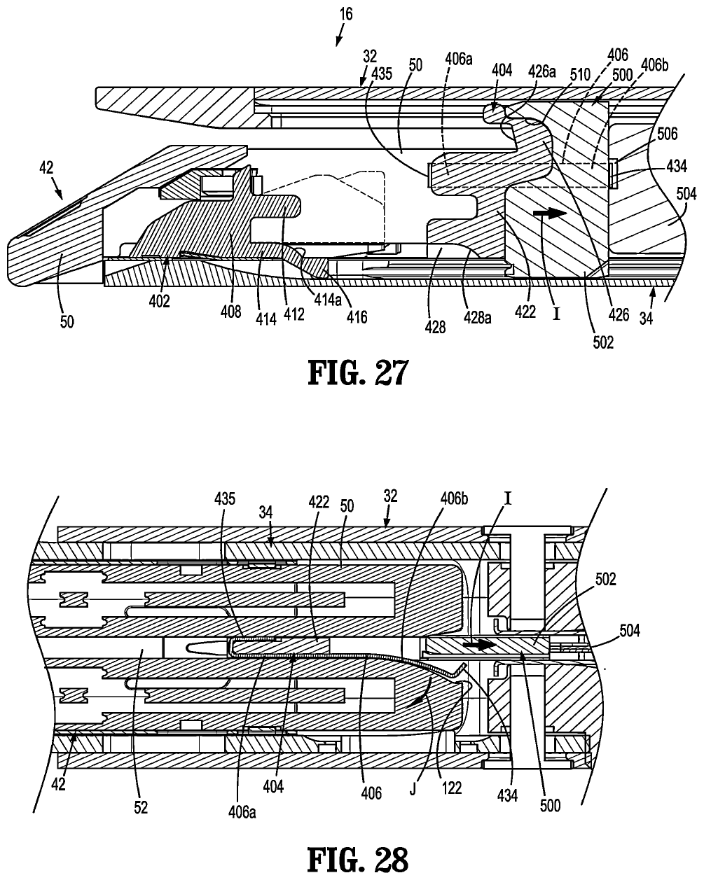

FIGS. 23 to 28 illustrate an alternate version of the actuation sled assembly and drive assembly of the stapling device 10 (FIG. 1) shown as actuation sled assembly 400 and drive assembly 500 (FIG. 27). The actuation sled assembly 400 includes an actuation sled 402, a knife bar 404, and a retractor link 406. The actuation sled assembly 400 is like the actuation sled assemblies 66 (FIG. 5) and 266 (FIG. 21) except that the knife bar 404 is not secured to the actuation sled 402. The actuation sled assembly 400 is received in the cartridge body 50 (FIG. 27) of the staple cartridge 42 (FIG. 4) and is movable within the cartridge body 50 between sled retracted and sled advanced positions. The actuation sled 402 includes a central portion 408 and spaced wedge members 410 that are positioned on opposite sides of the central portion 408. The wedge members 410 are configured to engage and lift the pushers 62 (FIG. 4) as the actuation sled assembly 400 is moved from the sled retracted position towards the sled advanced position to eject the staples 60 (FIG. 4) from staple receiving pockets 54 (FIG. 4) of the cartridge body 50. The central portion 408 of the actuation sled 402 is received in the knife slot 52 of the cartridge body 50 and is movable through the knife slot 52 to limit the actuation sled assembly 400 to linear movement. The central portion 408 of the actuation sled 402 defines a proximally extending finger 412, a longitudinally extending rib 414 (FIG. 24), and a member 416 that has a flat surface 416a. The rib 414 has a curved proximal surface 414a. The actuation sled 402 also defines a cutout 420 between the wedge members 410 that is dimensioned to receive the knife bar 404 as described below. The rib 414 and the member 416 are received in the cutout 420 and the flat surface 416a of the member 416 extends proximally of the cutout 420.

The knife bar 404 includes a body portion 422, a base portion 424, and knife 426. The body portion 422 of the knife bar 404 is received in and movable through the knife slot 52 of the cartridge body 50 (FIG. 4). The knife 426 includes a distally facing cutting edge 426a and is supported on an upper end of the body portion 422 of the knife bar 404 and projects proximally of a lower portion of the body portion 422 of the knife bar 404. The cutting edge 426a extends upwardly from the body portion 422 of the knife bar 404 to a position above the tissue engaging surface 50a (FIG. 4) of the cartridge body 50 and is shielded by the knife guards 58 (FIG. 4) when the knife bar 404 is in the retracted position. The base portion 424 is secured to or formed on the lower end of the body portion 422 and defines a channel 428 (FIG. 24) that is positioned to receive the rib 414 of the actuation sled 402. The base portion 424 extends outwardly of the body portion 422 and is positioned to ride along a bottom wall of the staple guard 64 (FIG. 4). The channel 428 in the base portion 424 of the knife bar 404 is defined in part by a curved proximal portion 428a (FIG. 27) that is configured to engage the curved proximal surface 414a of the rib 414 when the knife bar 404 is engaged with the actuation sled 402. The body portion 422 of the knife bar 404 defines a longitudinally extending recess 430 (FIG. 24) that receives a distal portion 406a of the retractor link 406. In aspects of the disclosure, the actuation sled 402 is formed of plastic and the knife bar 404 is formed from metal.

The retractor link 406 is secured to the body portion 422 of the knife bar 404 in cantilevered fashion. In aspects of the disclosure, the retractor link 406 is formed of a resilient material, e.g., spring steel, and includes the distal portion 406a and a proximal portion 406b. The proximal portion 406b of the retractor link 406 includes an engaging member which may be in the form of a 90-degree bend 434. The distal portion 406a defines a longitudinal axis and is secured to the central portion 408 of the knife bar 404 within the recess 430. In aspects of the disclosure, the distal portion 406a of the retractor link 406 has a u-shaped portion 435 that receives the body portion 422 of the knife bar 404. The proximal portion 406b of the retractor link 406 extends proximally from the knife bar 404. The retractor link 406 as described above regarding the retractor link 280 (FIG. 20) is movable between a non-deformed condition and a deformed condition. In the non-deformed condition, the proximal portion 406b of the retractor link 406 bends outwardly from the longitudinal axis defined by the distal portion 406a of the retractor link 406 and outwardly of the knife slot 52 (FIG. 4) of the cartridge body 50. When the actuation sled assembly 400 is advanced through the knife slot 52 of the cartridge body 50, the proximal portion 406b of the retractor link 406 engages an inner wall of the cartridge body 50 defining the knife slot 52 and is moved to the deformed condition in which the distal and proximal portions 406a and 406b of the retractor link 406 are longitudinally aligned.

The drive assembly 500 (FIG. 26) is like the drive assemblies 98 (FIG. 13) and 298 (FIG. 22) and includes a working member 502 and a flexible drive beam 504 (FIG. 27). The distal portion of the flexible drive beam 504 defines a recess or opening 506 (FIG. 27) that receives the bend 434 in the proximal portion 406b of the retractor link 406 when the retractor link 406 is deformed. The working member 502 of the drive assembly 500 defines a distally facing pocket 510 (FIG. 27) that receives the upper portion of the body portion 422 and knife 426 of the knife bar 404 (FIG. 26) when the working member 502 is moved into engagement with the knife bar 404.

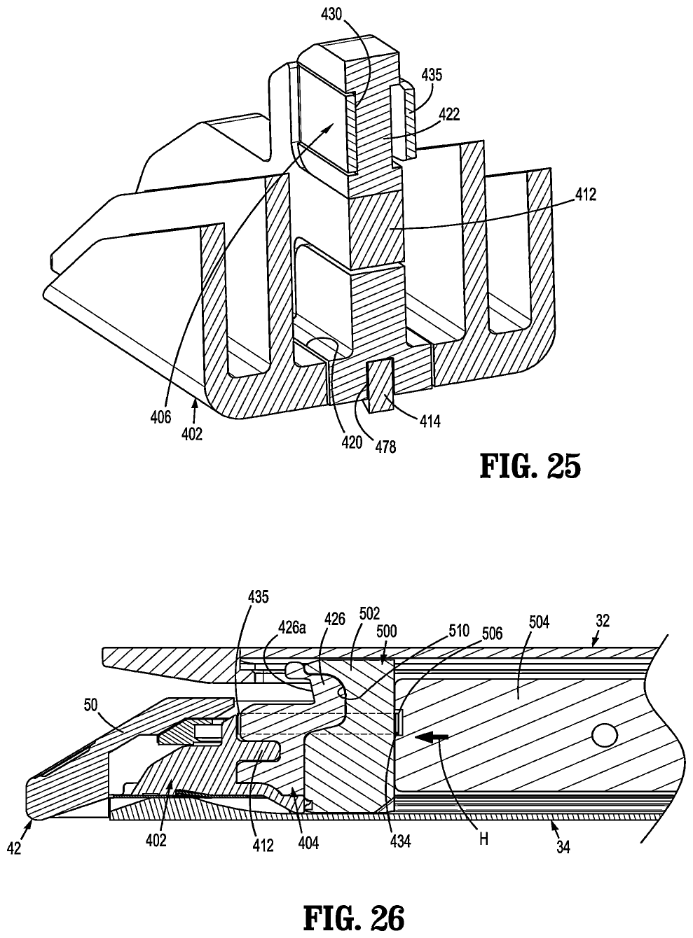

The actuation sled assembly 400 and the drive assembly 500 function in the same manner as the actuation sled assembly 266 and the drive assembly 298 described above except that the actuation sled 402 is only in abutting relation to the knife bar 404 and does not return to the retracted position with the knife bar 404 as the knife bar 404 is moved from the advanced position to the retracted position with the drive assembly 500. More specifically, when the drive assembly 500 moves from the drive assembly clamped position towards the drive assembly advanced position to move the actuation sled assembly 400 from the sled retracted position towards the sled advanced position, the retractor link 406 moves into the knife slot 52 (FIG. 4) and is deformed from the undeformed condition to the deformed condition. When this occurs, the bend 434 of the retractor link 406 is received in the opening 506 of the drive assembly 500 to couple the drive assembly 500 with the knife bar 404. As the drive assembly 500 moves towards the advanced position in the direction of arrow “H” in FIG. 26, the working member 502 of the drive assembly 500 engages the actuation sled 402 to move the actuation sled 402 from the sled retracted position to the sled advanced position to eject staples 60 (FIG. 4) from the staple cartridge 42. It is noted that when the working member 502 of the drive assembly 500 is engaged with the knife bar 404, the knife 426 of the knife bar 404 is received within the pocket 510 defined in the working end 502 of the drive assembly 500. In addition, when the knife bar 404 is engaged with the actuation sled 402, the base portion 424 of the knife bar 404 is received in the cutout 420 defined by the actuation sled 402. When the base portion 424 (FIG. 24) of the knife bar 404 is received in the cutout 420 (FIG. 25) of the actuation sled 402, the base portion 424 is supported on the flat surface 416a of the member 416 such that the rib 414 of the actuation sled 402 is received within the channel 428 defined in the base portion 424 of the knife bar 404.

FIG. 27 illustrates the distal portion of the tool assembly 16 (FIG. 1) as the drive assembly 500 is moved in the direction of arrow “I” from the advanced position back towards the retracted position. As the drive assembly 500 is retracted, the knife bar 404 which is coupled to the drive assembly 500 by the retractor link 406 also moves towards the retracted position. However, the actuation sled 402 which is only in abutting relation to the knife bar 404 disengages from the knife bar 404 and remains in the distal portion of the staple cartridge 42. (FIG. 27).

FIG. 28 illustrates the proximal portion of the tool assembly 16 as the drive assembly 500 approaches the retracted position. When the drive assembly 500 nears the retracted position, the proximal portion 406b of the retractor link 406 becomes aligned with the pocket 122 in the cartridge body 50 of the staple cartridge 42 and returns to the non-deformed condition in the direction of arrow “J” to disengage the knife bar 404 from the drive assembly 500. When this occurs, the drive assembly 500 continues to move to the retracted position independently of the knife bar 404. Disengaging the knife bar 404 from the drive assembly 500 allows for removal and replacement of the staple cartridge 42 to facilitate reuse of the stapling device 10 (FIG. 1).

Persons skilled in the art will understand that the devices and methods specifically described herein and illustrated in the accompanying drawings are non-limiting exemplary aspects of the disclosure. It is envisioned that the elements and features illustrated or described in connection with one exemplary embodiment may be combined with the elements and features of another without departing from the scope of the disclosure. As well, one skilled in the art will appreciate further features and advantages of the disclosure based on the above-described aspects of the disclosure. Accordingly, the disclosure is not to be limited by what has been particularly shown and described, except as indicated by the appended claims.

What is claimed is:

1. An actuation sled assembly for a surgical device, comprising:
an actuation sled comprising a central portion and one or more wedge members;
a knife bar coupled to the actuation sled; and
a retractor link comprising a spring bar having a proximal portion and a distal portion, with the distal portion secured to the knife bar and extending longitudinally;
wherein the retractor link defines:
a non-deformed condition in which the proximal portion bends away from the distal portion and the knife bar, and
a deformed condition in which the proximal portion is longitudinally aligned with the distal portion, with the retractor link biased toward the non-deformed position.

2. The actuation sled assembly of claim 1, wherein the knife bar comprises a longitudinally extending recess, with the distal portion of the retractor link secured within the longitudinally extending recess.

3. The actuation sled assembly of claim 1, wherein the proximal portion of the retractor link comprises an engaging member configured to engage a drive assembly of the surgical device, the engaging member comprising one of a 90-degree bend or a rounded protrusion.

4. The actuation sled assembly of claim 3, wherein the engaging member is disposed on a side of the retractor link facing the knife bar.

5. The actuation sled assembly of claim 3, wherein the engaging member of the retractor link is configured to engage the drive assembly when moving to the deformed condition.

6. The actuation sled assembly of claim 5, wherein the engaging member of the retractor link is configured to disengage from the drive assembly when moving to the non-deformed condition.

7. The actuation sled assembly of claim 1, wherein the knife bar comprises:
a body secured to the actuation sled; and
a knife carried by the body and having a distally facing cutting edge.

8. The actuation sled assembly of claim 6, wherein the central portion of the actuation sled comprises a cutout for slidable insertion of the knife bar.

9. The actuation sled assembly of claim 1, wherein the proximal portion of the retractor link extends proximally beyond the actuation sled.

10. A staple cartridge for a surgical device, comprising:
a cartridge body defining a knife slot;
a plurality of staples carried by the cartridge body; and
an actuation sled assembly disposed within the cartridge body and comprising:
an actuation sled comprising a central portion and one or more wedge members;
a knife bar coupled to the actuation sled and movable through the knife slot; and
a retractor link comprising a spring bar having a proximal portion and a distal portion, with the distal portion secured to the knife bar and extending longitudinally;
wherein the retractor link defines:
a non-deformed condition in which the proximal portion bends away from the distal portion and the knife bar, and
a deformed condition in which the proximal portion is longitudinally aligned with the distal portion, with the retractor link biased toward the non-deformed position.

11. The staple cartridge of claim 10, wherein the cartridge body further comprises a curved inner wall defining a pocket, and wherein the retractor link is configured to extend into the pocket when in the non-deformed condition.

12. The staple cartridge of claim 11, wherein the retractor link is configured to disengage the knife bar from a drive assembly of the surgical device when moving to the non-deformed condition and into the pocket.

13. The staple cartridge of claim 12, wherein the retractor link is configured to engage the knife bar with the drive assembly of the surgical device when moving to the deformed condition and out of the pocket.

14. The staple cartridge of claim 10, wherein the proximal portion of the retractor link extends proximally beyond the actuation sled.

15. A surgical stapling device, comprising:
a tool assembly, comprising:
an anvil;
a staple cartridge having a cartridge body defining a knife slot, a plurality of staples carried by the cartridge body, and an actuation sled assembly disposed within the cartridge body, the actuation sled assembly comprising:
an actuation sled comprising a central portion and one or more wedge members;
a knife bar coupled to the actuation sled and movable through the knife slot; and
a retractor link comprising a spring bar having a proximal portion and a distal portion, with the distal portion secured to the knife bar and extending longitudinally, wherein the retractor link defines a non-deformed condition in which the proximal portion bends away from the distal portion and the knife bar, and a deformed condition in which the proximal portion is longitudinally aligned with the distal portion, with the retractor link biased toward the non-deformed position; and
a drive assembly selectively coupled with the knife bar and movable between a retracted position and an advanced position to eject the plurality of staples;
wherein the retractor link is configured to disengage the knife bar from the drive assembly when in the non-deformed condition.

16. The surgical stapling device of claim 15, wherein the knife bar comprises a longitudinally extending recess, with the distal portion of the retractor link secured within the longitudinally extending recess.

17. The surgical stapling device of claim 15, wherein the proximal portion of the retractor link comprises an engaging member configured to selectively engage the drive assembly, the engaging member comprising one of a 90-degree bend or a rounded protrusion.

18. The surgical stapling device of claim 15, wherein the cartridge body further comprises a curved inner wall defining a pocket, and wherein the retractor link is configured to extend into the pocket when in the non-deformed condition.

19. The surgical stapling device of claim 18, wherein the retractor link is configured to move into the pocket to the non-deformed condition, disengaging the knife bar from the drive assembly, when the drive assembly moves to the retracted position.

20. The surgical stapling device of claim 19, wherein the retractor link is configured to move out of the pocket to the deformed condition, engaging the knife bar with the drive assembly, when the drive assembly moves to the advanced position.
